(19) United States
(12) Patent Application Publication
(10) Pub. No.: US 2021/0093049 A1
(43) Pub. Date: Apr. 1, 2021

(54) METHOD, DEVICE AND SYSTEM FOR MEASURING, EVALUATING AND SIMULATING A SHOE

(71) Applicant: MOLIBSO ENTWICKLUNGS- UND VERTRIEBS GMBH, LANGENFELD (DE)

(72) Inventor: JENS HOLLENBACHER, LANGENFELD (DE)

(73) Assignee: MOLIBSO ENTWICKLUNGS- UND VERTRIEBS GMBH, LANGENFELD (DE)

(21) Appl. No.: 16/981,700

(22) PCT Filed: Mar. 18, 2019
(86) PCT No.: PCT/EP2019/000083

§ 371 Date: Sep. 17, 2020

(30) Foreign Application Priority Data
Mar. 20, 2018 (DE) 10 2018 002 283.9
Mar. 20, 2018 (DE) 10 2018 002 284.7

(51) Int. Cl. A43D 1/02; A61B 5/103; A61B 5/107

(52) U.S. Cl. CPC A43D 1/02; A61B 5/1036; A43D 2200/60; A61B 2562/0247; A61B 5/1074

(57) ABSTRACT

A method for measuring and evaluating functions and a shape of a shoe. The method includes providing a measuring surface with pressure sensors, acquiring pressure values with the pressure sensors via a user wearing the shoe on the user's foot standing on, walking on, and/or running on the measuring surface, evaluating the pressure values acquired by the pressure sensors via a computer program, and determining a shape, a dimension and/or a material of a bottom of the shoe based on the pressure values acquired.

### CROSS REFERENCE TO PRIOR APPLICATIONS

This application is a U.S. National Phase application under 35 U.S.C. § 371 of International Application No. PCT/EP2019/000083, filed on Mar. 18, 2019 and which claims benefit to German Patent Application No. 10 2018 002 283.9, filed on Mar. 20, 2018, and to German Patent Application No. 10 2018 002 284.7, filed on Mar. 20, 2018. The International Application was published in German on Sep. 26, 2019 as WO 2019/179655 A1 under PCT Article 21(2).

### FIELD

The present invention relates to a method for measuring and evaluating the functions and the shape of a shoe, in particular of a sports shoe such as a running shoe or a ski boot. The present invention also relates to a method for generating data for the manufacturing of a shoe that is individually adapted to a person. The present invention also relates to a system for simulating a shoe that can be used to generate data for the manufacturing of shoes. The present invention also relates to a shoe simulation apparatus that exhibits the system and with which the method can be carried out.

### BACKGROUND

It is known to place pressure sensors inside a shoe to detect the different pressures applied by the plantar surface of the foot. The pressure sensors are located either in the upper side of the shoe bottom or in an insole. It is necessary to equip shoes or insoles with sensors in order to obtain the desired measured values. This not only makes manufacturing costly and time-consuming, but also changes the shape and dimensions of the shoe so that usable measurement results can hardly be achieved.

In the manufacturing of shoes, methods are increasingly being used which make it possible to individually adapt components of the shoes to a person and thus improve the fit of the resulting shoes. Shaped parts can in particular be designed to fit perfectly using 3D printing techniques.

It is known to adapt certain parts of shoes to the individual needs of a person based on measured values. DE 102016 009 980 A1 describes, for example, to adapt a molded foam component, in particular the shoe bottom of a sports shoe, to a person by measuring a pressure distribution of at least one foot of a person via a pressure measuring plate and adjusting the material properties of the foam material in three-dimensions based on the determined distribution of the pressure.

When manufacturing of components based exclusively on measured values, the person only has an opportunity to experience the feel of the shoe after the shoe has already been manufactured. The person in particular does not have any direct influence on the final product. It may thus happen that the person is not satisfied with the manufactured shoe because it deviates from the person's wishes.

### SUMMARY

An aspect of the present invention is to provide a method and a system that is as simple and as practical as possible, which can be used in a store, and which makes it possible to generate data on the basis of which a shoe can be manufactured that corresponds to the person's wishes.

An aspect of the present invention is also to provide a method for measuring and evaluating the functions and the shape of a shoe, in particular of a sports shoe, which provides measured values of shoes, in particular of sports shoes, without having to modify the shoes/sports shoes.

In an embodiment, the present invention provides a method for measuring and evaluating functions and a shape of a shoe. The method includes providing a measuring surface comprising a plurality of pressure sensors, acquiring pressure values with the plurality of pressure sensors via a user wearing the shoe on the user's foot at least one of standing on, walking on, and running on the measuring surface, evaluating the pressure values acquired by the plurality of pressure sensors via a computer program, and determining at least one of a shape, a dimension and a material of a bottom of the shoe based on the pressure values acquired.

### BRIEF DESCRIPTION OF THE DRAWINGS

The present invention is described in greater detail below on the basis of embodiments and of the drawings in which:

FIG. 1 is a representation of the system of the present invention;

FIG. 2 is a front view of a shoe simulation apparatus according to the present invention loaded by the foot of a person;

FIG. 3 is a top view of a shoe simulation apparatus according to the present invention loaded by the foot of a person with a grid of square resting sections; and

FIG. 4 is a is a top view of a shoe simulation apparatus according to the present invention loaded by the foot of a person with resting sections, the shapes of which are adapted to selected zones of the foot.

### DETAILED DESCRIPTION

The shape, dimensions and/or materials of the shoe bottom are determined based on the pressure values acquired.

The shape and the dimensions of the shoe upper can also be determined based on the pressure values acquired.

Any shoe can therefore be measured, evaluated and optimally manufactured without having to equip the shoe with sensors. The following properties can be identified and considered for any kind of shoe:
Does the shoe fit the shape of the respective foot?
Does the shoe match the individual movement of the user?
Does the user show signs of incorrect walking/running?
Which shape and dimensions should the shoe bottom have, in particular the outsole and/or the upper side of the shoe?
Which mechanical properties should the shoe bottom and/or the shoe upper have?
Which materials should be used for the shoe bottom and/or the shoe upper?
What is the wear and tear behavior of the shoe?

The method according to the present invention also comprises the following steps:
A measurement step, in which the plantar pressure distribution of at least one foot of the person is measured by a pressure distribution measuring apparatus on a measuring surface;
An adjustment step, in which a shoe simulation apparatus having a shoe bottom with a plurality of resting sections, the height and/or at least one mechanical property of which can each be individually controlled, is adjusted locally by adjusting the height and/or the at least one mechanical property of the resting sections to values that are determined by a computing unit on the basis of the measured pressure distribution; and
A simulation step, in which the person puts load on the shoe simulation apparatus in order to sense the properties of a shoe that exhibits the adjusted values.

In the initial measurement step, the pressure distribution of the plantar surface of the foot of the person is determined by a pressure distribution measuring apparatus. The pressure distribution can be measured statically, i.e., with the person standing still, or dynamically, i.e., with the person walking or running. The shoe simulation apparatus is then adjusted based on the determined pressure distribution. To sense the properties of a shoe exhibiting the adjusted properties, the person uses the shoe simulation apparatus by putting a load on the shoe bottom or the insole. The loading of the shoe simulation apparatus can be made statically, i.e., with the person standing still, or dynamically, i.e., with the person walking or running. Aided by the shoe simulation apparatus, the person senses the shoe bottom with those properties that the shoe will have after the shoe has been manufactured. The advantage thereof is that the person knows the properties of the shoe when buying the shoe so that mispurchases can be avoided.

For simulating a shoe, the shoe simulation apparatus has a shoe bottom which in turn exhibits a plurality of resting sections, each of the resting sections being separately controllable in their height and in at least one physical property. The profile of the shoe bottom can be individually adjusted by changing the height of the individual resting sections. Based on the pressure distribution it can, for example, be determined which sections of the shoe bottom are loaded more by the person so that, for these sections, a lower height is adjusted. The resting sections are designed to allow a separate and independent adjustment of the mechanical properties of each of the resting sections. A shoe bottom can thereby be simulated that has mechanical properties that are similar to those of the manufactured shoe.

The shoe simulation apparatus can alternatively comprise an insole which in the intended use is inserted into a shoe of a defined type and which exhibits a plurality of adjustable resting sections. An individually adapted insole for a defined shoe can thereby be manufactured. An individually adapted shoe can thereby be realized with an individually adapted insole as a component of a shoe.

The controllable mechanical properties of the resting sections of the shoe bottom and the insole are hardness, flexibility and elasticity. Control of the hardness is in particular a controllable mechanical property.

An embodiment of the method features an additional examination step in which the person can, for example, examine whether or not the locally adjusted values of the shoe simulation apparatus reflect the personal optimum or wish. If the currently adjusted values of the height and/or the mechanical properties of the resting sections do not match the desired adjustment, another adjustment step is performed with changed adjusted values. Whether or not the adjustments correspond to the personal optimum can be entered by the person and/or a third party, for example, an employee of the store, at the computing unit via an operator control unit. A mobile input device can, for example, be used that is connected with the computing unit by radio. The calculation of new, changed values of the height and/or the mechanical properties is performed by the computing unit, which calculates new values to be adjusted for essentially all resting sections as a substitute for the currently adjusted values. Preset parameters can, for example, be selected via which the person can select a generally softer or harder adjustment of the resting sections. The selection can, for example, be made at the input device.

The shoe simulation apparatus can, for example, be executed so that the person uses it in the adjustment step, i.e., puts load on the shoe bottom. The advantage of performing the adjustment step with the shoe bottom under load is that the person immediately senses the change so that it is easier for the person to identify whether or not a change is made in the desired direction.

Manufacturing, measurement and evaluation are significantly improved if the data/measured data of a known optimum shoe are acquired and this data is compared with the data measured by one of the above methods and utilized accordingly.

In an embodiment of the method of the present invention, an optimization step can, for example, be performed in which the height and/or the at least one mechanical property of the resting sections are each adjusted locally using an input device. The person or a third party, for example, an employee of the store, is thereby able to use an input device for the local adjustment of the values of the individual resting sections and hence individually modify the shoe bottom. The input device can be a mobile terminal with a software application which exchanges data with the computing unit by radio. In the optimization step, the shoe simulation apparatus may also be used, i.e., with the shoe bottom under load, or may not be used, i.e., with the shoe bottom not under load. The adjustable local change of the properties of the resting sections is particularly advantageous for persons for whom a local modification of the height profile and/or the mechanical properties is required for orthopedic or medical grounds. This can be advantageous, for example, for people with neurological disorders such as diabetes to set certain stimuli on the plantar surface of the foot in order to improve the sense of self-movement and body position (proprioception) or to optimize the pressure distribution/relief.

The method can, for example, comprise using at least one holding device which is provided at the simulation apparatus to locate the foot of the person on the shoe bottom, the holding device being adjusted in its length so as to be adapted to the anatomy of the person. The shoe simulation apparatus may thereby be used to simulate not only the shoe bottom or the insole, but also the shoe upper so that the person senses the simulated shoe as a contiguous unit. The exact design of the holding device may vary depending on the type of the shoe. At least one of the holding devices can, for example, comprise a contraction element, the length of which can be set to a desired value via the computing unit, via the input, and/or manually. The length of the respective holding device can be specifically controlled and specifically registered for the respective person. Controlling the length of the contraction element via the input device has the advantage that the person can specifically control the length of the holding device and can, for example, do so when using the shoe simulation apparatus, i.e., when putting a load on the shoe bottom or the insole. The person can thereby immediately experience the effect of a change of the lengths of individual holding devices. In particular in combination with the local change of the values of the height and/or the mechanical properties of the resting sections, individually optimized adjustments to fulfil the wish of the person can thereby be determined. The specific adjustment of the lengths of the holding device can, for example, have an orthopedic-engineering effect.

In an embodiment of the method of the present invention, the values of the height and/or the at least one mechanical property of the resting sections and/or the length of the holding device which the person considers to be the optimal values can, for example, be stored in a memory unit. The optimum adjustments for a person can thereby be registered and can be reproduced at any place and time. A shoe simulation apparatus located at a different place may, for example, be controlled with the stored values so that the adjustment for a person can be experienced at a different place. The person is also able to sense the simulated shoe at a later time, for example, if the person decides only later to make a purchase or a purchase decision.

In an embodiment of the present invention, the stored values can, for example, be used as data for the manufacturing of individually adapted shoes. The availability of the values of the height and/or the at least one mechanical property of the resting sections and/or the length of the holding device in form of the stored data makes it possible to manufacture the shoe considered to be optimal at a different place. There are thus no differences between the produced shoe and the simulated shoe except for manufacturing tolerances. This will, for example, avoid mispurchases and save resources.

The measurement step of the underlying method can, for example, additionally comprise the determination of the three-dimensional shape of the foot of a person via at least one scanning apparatus. By using a scanning apparatus, additional information is obtained that can be directly used to determine the heights of the resting sections, or the profile of the shoe bottom, or the insole, respectively. Commercial 3D scanners can be used as the scanning apparatus. The shape of the foot can, for example, be measured when the foot is located freely in space, i.e., is not loaded.

In an embodiment of the method of the present invention, a display unit can, for example, be used to optically suggest to the person using the shoe simulation apparatus that the person is wearing a shoe exhibiting an outer shape (design, color, symbols) that the person selects or has selected. Such a display unit may be a monitor which the person views and on which a certain shoe is displayed at the location of the feet. The display unit can, for example, comprise virtual reality glasses in which the shoes are displayed at the place where the feet of the person are located. The immersion, i.e., the perception of the simulated environment as reality, is thereby improved and a perception of the simulated shoe as a real shoe is accomplished. An outer appearance of the shoe can, for example, be shown which the customer has designed or designs himself/herself, so that a personalization is also feasible and perceivable as regards the outer shape.

The present invention also relates to a system for the simulation of a shoe when manufacturing a shoe that is individually adapted to a person, the system comprising:
A pressure distribution measuring apparatus which is designed to determine a plantar pressure distribution of at least one foot of the person;
A computing unit which is designed to evaluate the determined pressure distribution; and
A shoe simulation apparatus which exhibits a shoe bottom or an insole with a plurality of resting sections, the height and/or at least one mechanical property of which can each be individually controlled by the computing unit on the basis of the determined pressure distribution.

Such a system can be used to implement the method described above and hence be used to manufacture a shoe that is individually adapted to a person. The system comprises components that have a small footprint so that this system can, for example, be used in stores.

The pressure distribution measuring apparatus of the system can, for example, be a pressure measuring plate, a pressure measuring sheet, or a sensitive insole. A pressure measuring plate can, for example, exhibit a local resolution and pressure sensitivity which make it possible to determine the plantar pressure distribution both statically, i.e., with the person standing still, and dynamically, i.e., with the person walking or running. Capacitive pressure measuring plates can, for example, be used.

In an embodiment of the present invention, the system can, for example, comprise at least one scanning apparatus that is designed to determine the three-dimensional shape of the foot. Using scanning systems is a simple way to determine the foot's shape. The data generated in this way can be directly used for the determination of the values of the heights of the resting sections, or the shoe bottom profile, respectively.

The system can, for example, comprise an input device that is designed to adjust the height and/or at least one mechanical property of each of the resting sections depending on the respective position. The values can thereby be modified and optimized locally by the person or a third party. The input device can also be designed, for example, to make a parameter selection in the adjustment step.

In an embodiment of the present invention, the system can, for example, comprise a display unit that is designed to give the user the impression that the user is wearing a shoe of a particular type or design. The design can, for example, be selected by the person so that the desired execution of the shoe can also be simulated regarding the outer design. The person can, for example, design the outer appearance himself/herself.

The system can, for example, comprise a movably supported shoe simulation apparatus. The natural walking movement or weight shifts at the foot can thereby be tracked by the shoe simulation apparatus. This can, for example, generate a realistic sensory experience. A mobile execution of the simulation apparatus can, for example, be provided so the person can walk or run around therewith. The shoe simulation apparatus is in this case, for example, connected with the computing unit by radio.

In an embodiment of the present invention, the shoe simulation apparatus can, for example, be separated from the system while the adjusted values of the height and/or the at least one mechanical property are retained. This is particularly advantageous if the shoe simulation apparatus comprises an insole. The person can thereby reuse an insole with optimally adjusted values of the height and/or the at least one mechanical property. This is particularly advantageous if the insole is inserted during the adjustment in a shoe that the person wishes to continue to wear afterwards.

The present invention also relates to a shoe simulation apparatus that is designed to accommodate a foot of a person. The shoe simulation apparatus comprises a shoe bottom or an insole having a plurality of resting sections, the height and/or at least one mechanical property of which can each be individually controlled. The resting sections can have any basic shape. In an embodiment, the individual resting sections can, for example, form a grid in which all resting sections have the same basic shape, e.g., squares or circles. In an embodiment, the resting sections can, for example, have ergonomically adapted shapes which mirror individual zones of the foot, e.g., an elliptical shape for the heel area or a kidney shape for the ball. This allows a better adaptation of the values of the mechanical properties of the resting sections to the respective function.

The shoe simulation apparatus can, for example, comprise at least one holding device that is adjustable in its length and is designed to clasp the foot of the person and locate it on the shoe bottom. This in particular makes it possible to simulate the shoe upper and make it perceivable for the person. In an embodiment of the present invention, at least one holding device can, for example, have at least one contraction element, the length of which can be adjusted via a computing unit and/or via an input device and/or manually.

In an embodiment of the present invention, the resting sections of the shoe simulation apparatus each exhibit sensors that are designed to determine a force applied on the respective resting section. It is thereby possible to use the shoe simulation apparatus itself as a sensor unit which can be used to check the adjustment of the height and/or the mechanical properties and/or the length of the holding device. For example, load peaks of a particular resting section can be identified when this section measures a force that is excessive compared with the surrounding resting sections. An automatic monitoring of the adjusted values can thereby already be performed during the simulation step, the examination step and/or the optimization step.

Embodiments of the present invention are shown schematically in the drawings which are explained in greater detail below.

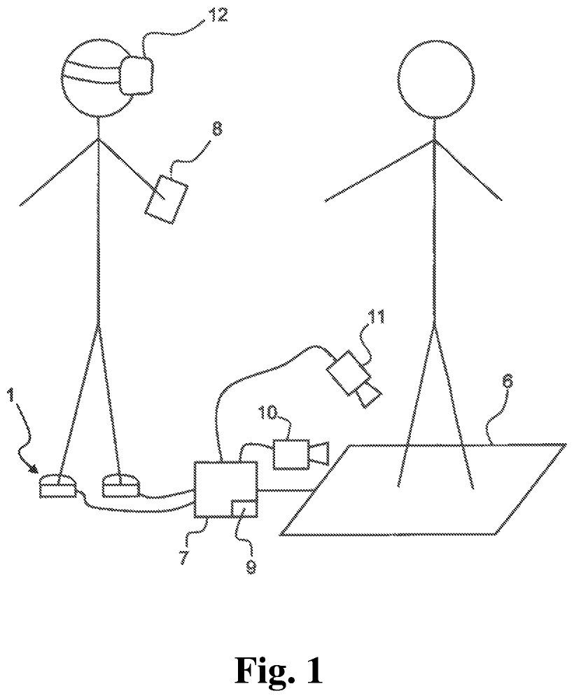

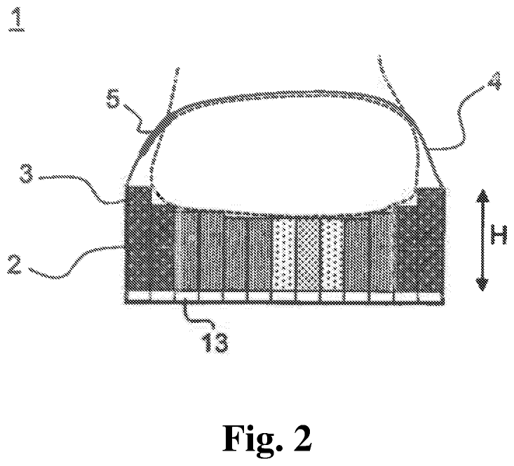

FIG. 1 shows a system for simulating a shoe, which system can be used to generate data for the manufacturing of shoes or insoles that are individually adapted to a person. The system comprises a pressure distribution measuring apparatus 6 that is designed to determine the plantar pressure distribution of at least one foot of a person, the pressure distribution measuring apparatus 6 being a pressure measuring plate, a pressure measuring sheet or a sensitive insole. The measured pressure distribution is evaluated via a computing unit 7. The system also comprises a shoe simulation apparatus 1 which is designed to simulate a shoe. For this purpose, the shoe simulation apparatus 1 according to FIG. 2 exhibits a shoe bottom 2 or an insole with a number of resting sections 3 the height H and/or at least one mechanical property of which can each be individually controlled by means of the computing unit 7 on the basis of the determined pressure distribution.

For the determination of the three-dimensional shape of at least one foot of the person, the system can, for example, comprise two scanning apparatuses 10 and 11. This makes it possible to directly determine the profile of the shoe bottom 2, or the heights of the individual resting sections 3, respectively. Optical 3D scanners can, for example, be used therefor. The scanning apparatuses 10 and 11 are connected with the computing unit 7 by cable or radio for the exchange of data.

The system also comprises an input device 8 that is designed to adjust the height and/or at least one mechanical property of each of the resting sections 3 depending on the respective position. The person can thereby locally optimize the adjustments of the simulation apparatus 1 to realize desired properties.

The system also comprises a display unit 12 that is designed to give the user the impression that he or she is wearing a particular shoe. This display unit 12 is comprised of virtual reality glasses 12 worn by the person while using the simulation apparatus 1, thereby giving the impression that the person is wearing a particular shoe. The displayed shoe can be a shoe of a certain type or design. A shoe may be in particular displayed that the person has selected or designed himself/herself. The outer appearance of the shoe can thereby be simulated and personalized.

The computing unit 7 has a memory unit 9 which is designed to store the adjusted values of the height and/or the at least one mechanical property of the resting sections 3 and/or the length of a holding device 4. The values considered by the person to be optimal or to be as desired are in particular stored. These values can then be used to reproduce a defined adjustment, in particular, an adjustment that is as desired. The stored values that are considered to be optimal are in particular used as data for manufacturing an individually adapted shoe, the manufactured shoe having the same properties as the shoe simulated by the shoe simulation apparatus 1.

In an embodiment of the present invention that is not shown, the shoe simulation apparatus 1 can, for example, be movably supported. The shoe simulation apparatus 1 can, for example, be mobile so that the person can walk or run around therewith.

FIG. 2 shows a schematic representation of the shoe simulation apparatus 1 when it is used, i.e., loaded, with the person standing on the shoe bottom 2. The values of the height H of the individual resting sections 3 are adjusted so that the height profile of the shoe bottom 2 matches the three-dimensional shape of the plantar surface of the foot. The different textures of the individual resting sections 3 indicate that the resting sections 3 each have different mechanical properties. For example, the outer resting sections 3 exhibit a lower elasticity in order to provide the foot with a better hold when it is exposed to a load.

The shoe simulation apparatus 1 comprises at least one holding device 4 that is designed to clasp the foot of the person and locate it on the shoe bottom 2. The holding device can, for example, be a flat tape which is attached with its end points at the shoe bottom 2. The length of the shown holding device 4 can be changed via a contraction element 5. The length is adjusted by the computing unit 7 and/or by the input device 8 or manually. The adjustment of the length can be made by the person or by a third party. The length of the contraction element 5 of a simulated shoe can, for example, be stored in the computing unit 7 so that the length can be reproduced at another time and place.

The resting sections 3 also each have sensors 13 that are designed to measure a force applied on the respective resting section 3. The shoe simulation apparatus 1 thus comprises a sensor unit 13 that makes it possible to directly verify the adjusted values in the loaded condition. Undesired force peaks, or pressure peaks, respectively, can therefore, for example, be directly registered.

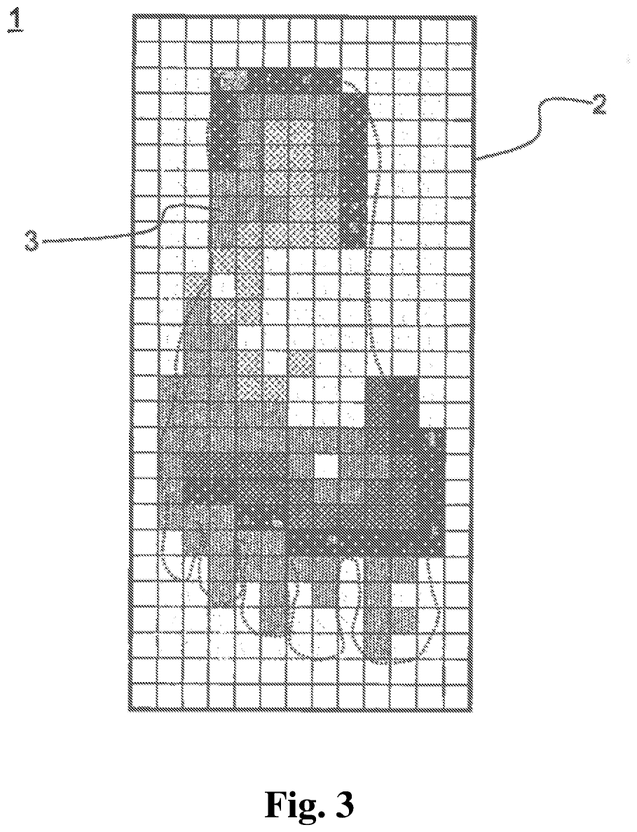

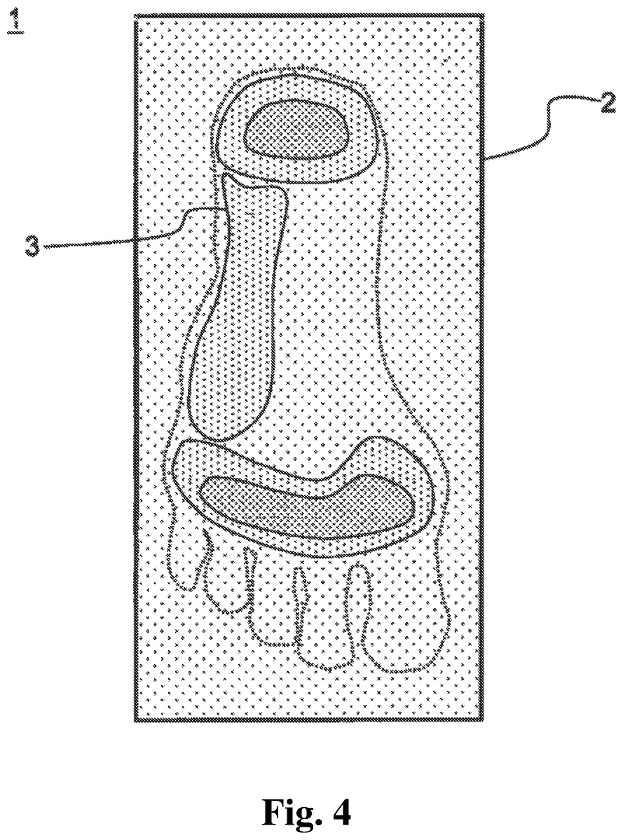

The resting sections 3 of the shoe bottom 2 of the shoe simulation apparatus 1 can have any shape. FIG. 3 shows an embodiment in which the individual resting sections 3 form a grid with all resting sections 3 having the same square basic shape. The foot of the person is shown as a broken line. The different textures of the individual resting sections 3 schematically show that the individual resting sections 3 exhibit different mechanical properties that can be adjusted independent from one another. The outer resting sections 3 exhibit a lower elasticity to better support the foot. FIG. 4 shows an alternative embodiment in which the resting sections 3 have ergonomically adapted shapes that imitate individual zones of the foot. For example, the resting sections 3 in the heel area may have an elliptical shape and the sections in the ball area may have kidney shape. This allows a better adaptation of the values of the mechanical properties of the resting sections 3 to the respective function.

For the method according to the present invention, a measuring surface or a measuring plate is also used that has a plurality of sensors that are located closely next to one another in a grid layout. The sensor areas or sensor area sections are located planarly in an x and y direction in a matrix. If this measuring surface is touched by a shoe with its bottom side, i.e., its outsole, a plurality of pressure sensors are touched and exposed to a more or less strong pressure. Every pressure sensor transduces the physical quantity pressure into an electrical quantity (voltage). These electrical values/signals are acquired and stored by a computer. A computer program then displays the acquired values graphically and in color so that it can be seen which locations of the outsole apply a stronger pressure and which apply a weaker pressure on the measuring surface.

Both when the person is standing still with the person's shoe or shoes on the measuring surface and when the persons walks or runs over the measuring surface, valuable measurement results in the form of graphical images and videos are created which lead to the above-mentioned effects and advantages.

Pressure sensors of various kind can be used, both passive and active pressure sensors. Capacitive, piezoresistive and piezoelectric pressure sensors can, for example, be used.

The measuring surface/measurement plate is, for example, not square, but exhibits a greater length than width so that it is possible to walk/run thereon in its longitudinal direction. In this case, the measuring surface is two-dimensional planar.

The measuring surface is alternatively three-dimensional curved/cambered to simulate a natural running track. The measuring surface can also represent a gradient by forming an inclined level.

The data required for the evaluation and manufacturing are further improved if the data/measured data of a known optimal shoe is acquired and this data is compared with the data measured by one of the above methods and utilized accordingly.

The present invention is not limited to embodiments described herein; reference should be had to the appended claims.

What is claimed is:

1-26. (canceled)

27. A method for measuring and evaluating functions and a shape of a shoe, the method comprising:
providing a measuring surface comprising a plurality of pressure sensors;
acquiring pressure values with the plurality of pressure sensors via a user wearing the shoe on the user's foot at least one of standing on, walking on, and running on the measuring surface;
evaluating the pressure values acquired by the plurality of pressure sensors via a computer program; and
determining at least one of a shape, a dimension and a material of a bottom of the shoe based on the pressure values acquired.

28. The method as recited in claim 27, further comprising:
determining at least one of a shape and a dimension of a shoe upper of the shoe based on the pressure values acquired.

29. A method for generating data for at least one of a manufacturing, a measurement, and an evaluation of a shoe that is individually adapted to a person, the method comprising:
providing a pressure distribution measuring apparatus comprising a measuring surface;
providing a shoe simulation apparatus comprising,
a shoe bottom which comprises a plurality of resting sections, wherein at least one of a height and at least one mechanical property of the plurality of resting sections is individually controllable, or
an insole which comprises a plurality of resting sections (3), wherein at least one of a height and at least one mechanical property of the plurality of resting sections is individually controllable;
a measurement step comprising measuring a plantar pressure distribution of at least one foot of the person via the pressure distribution measuring apparatus on the measuring surface;
an adjustment step comprising locally adjusting the shoe simulation apparatus by adjusting at least one of the height and the at least one mechanical property of the plurality of resting sections to adjusted values determined by a computing unit based on the determined plantar pressure distribution; and
a simulation step comprising simulating properties of the shoe with the adjusted values via putting a load on a bottom of the shoe.

30. The method as recited in claim 29, further comprising:
an optimization step comprising optimizing by locally adjusting at least one of the height and the at least one mechanical property of the plurality of resting sections via an input device.

31. The method as recited in claim 29, wherein,
at least one holding device is arranged in the shoe simulation apparatus, the at least one holding device being configured to locate the foot on a bottom of the shoe,
wherein the method further comprises:
adjusting a length of the at least one holding device.

32. The method as recited in claim 31, wherein the length of the at least one holding device is adjusted via a contracting device.

33. The method as recited in claim 29, further comprising:
an examination step comprising a person examining whether the adjusted values of the shoe simulation apparatus reflect a personal optimum or whether an additional locally adjusting step with changed adjustment values is required.

34. The method as recited in claim 33, further comprising:
storing in a memory unit the adjusted values of at least one of the height and the at least one mechanical property of the plurality of resting sections which the person considers to be optimal values.

35. The method as recited in claim 34, further comprising;
manufacturing individually adapted shoes using the optimal values stored in the memory unit.

36. The method as recited in claim 34, further comprising:
acquiring data of an optimum shoe; and
comparing the data from the optimum shoe with at least one of the adjusted values, the changed adjusted values, and the optimal values.

37. The method as recited in claim 29, wherein the measurement step further comprises:
determining a three-dimensional shape of the foot of the person via at least one scanning apparatus.

38. The method as recited in claim 29, further comprising:
providing a display unit to optically suggest to the person using the shoe simulation apparatus that the person is wearing a shoe having a particular type or design

39. The method as recited in claim 38, wherein the shoe having the particular type or design is a design created by the person.

40. A system for a simulation of a shoe to generate data for manufacturing a shoe that is individually adapted to a person via the method as recited in claim 29, the system comprising:
the pressure distribution measuring apparatus which is configured to determine a plantar pressure distribution of at least one foot of the person;
the computing unit which is configured to evaluate the determined plantar pressure distribution; and
the shoe simulation apparatus which comprises:
the shoe bottom comprising the plurality of resting sections, wherein at least one of the height and the least one mechanical property of the plurality of resting sections is individually controllable via the computing unit based on the plantar determined plantar pressure distribution, or
the insole comprising the plurality of resting sections, wherein at least one of the height and the at least one mechanical property of the plurality of resting sections is individually controllable via the computing unit based on the determined plantar pressure distribution.

41. The system as recited in claim 40, wherein the pressure distribution measuring apparatus is a pressure measuring plate, a pressure measuring sheet, or a sensitive insole.

42. The system as recited in claim 40, further comprising:
at least one input device which is configured to adjust at least one of the height and the at least one mechanical property of each of the resting sections depending on a respective position.

43. The system as recited in claim 40, further comprising:
at least one display unit which is configured to provide the person with an impression that the person is wearing a particular shoe.

44. The system as recited in any of claim 40, wherein the shoe simulation apparatus is configured to be movably supported.

45. The system as recited in claim 40, wherein the shoe simulation apparatus is configured to be separated from the system while the adjusted values of at least one of the height the at least one mechanical property are retained.

46. The system as recited in claim 45, wherein the insole of the shoe simulation apparatus is configured to be separated from the system while the adjusted values of at least one of the height the at least one mechanical property are retained.

47. The shoe simulation apparatus as recited in claim 40, the shoe simulation apparatus being configured to accommodate the at least one foot of the person, the shoe simulation apparatus comprising:
the shoe bottom comprising the plurality of resting sections, wherein at least one of the height and the least one mechanical property of the plurality of resting sections is individually controllable, or
the insole comprising the plurality of resting sections, wherein at least one of the height and the at least one mechanical property of the plurality of resting sections is individually controllable.

48. The shoe simulation apparatus as recited in claim 47, further comprising:
at least one holding device which is configured to be adjustable in a length thereof, to clasp the at least one foot of the person, and to locate the at least one foot of the person on the shoe bottom.

49. The shoe simulation apparatus as recited in claim 48, wherein,
each of the at least one holding device comprises at least one contraction element, and
a length of the at least one contraction element is configured to be adjustable via at least one of a computing unit, via an input device, and manually.

50. The shoe simulation apparatus as recited in claim 47, wherein the plurality of resting sections each comprise sensors which are configured to determine a force applied on the respective plurality of resting sections.

51. The measuring surface for use in the method as recited in claim 29, wherein the measuring surface is subdivided in a grid-like manner into a plurality of sensor area sections in a full-coverage layout.

52. The measuring surface as recited in claim 51, wherein the measuring surface is two-dimensional planar.

53. The measuring surface as recited in claim 51, wherein the measuring surface is at least one of three-dimensional curved and three-dimensional cambered.
